(19) United States
(12) Patent Application Publication
(10) Pub. No.: US 2026/0306300 A1
(43) Pub. Date: Oct. 8, 2026

(54) DEVICE FOR RETRIEVAL OF FOREIGN OBJECTS FROM BODILY CAVITIES

(71) Applicant: MOR RESEARCH APPLICATIONS LTD., Ramat Gan (IL)

(72) Inventors: Itzhak POMERANTZ, Ramat Gan (IL); Ella ELON WEISS, Ramat Gan (IL); Hila MILER, Ramat Gan (IL); Noam MEIR, Herzliya (IL)

(73) Assignee: MOR RESEARCH APPLICATIONS LTD., Ramat Gan (IL)

(21) Appl. No.: 19/479,432

(22) PCT Filed: May 1, 2024
(86) PCT No.: PCT/IL2024/050411

§ 371 Date: Oct. 28, 2025

Related U.S. Application Data
(60) Provisional application No. 63/463,093, filed on May 1, 2023.

(51) Int. Cl. A61F 11/00; A61B 17/00; A61B 17/24

(52) U.S. Cl. CPC A61F 11/006; A61B 17/24; A61B 2017/00296; A61B 2017/00951; A61B 2017/246

(57) ABSTRACT

The present disclosure provides a device for foreign object extraction from a cavity of a subject. The device comprises (a) an insertion portion configured for being inserted into the cavity; (b) a reservoir configured to contain a fluidic activable adhesive substance, and for delivering the activable adhesive substance to a facing surface of the foreign object; (c) an adhesive substance actuator configured to controllably activate the adhesive substance so as to transition the adhesive substance from a non-adhesive state to an adhesive state that generates bonding to the foreign object.

### TECHNOLOGICAL FIELD

The presently disclosed subject matter generally relates to the field of medical devices, particularly to a disposable device designed for the extraction of foreign objects from bodily cavities (e.g. aural or nasal cavities) using a controllably activable adhesive substance.

### BACKGROUND

The extraction of foreign objects from aural (e.g. ear canal) and nasal cavities is a delicate medical procedure. Traditional removal methods typically involve the use of mechanical grasping tools, such as forceps, hooks, or suction devices. These methods can be challenging due to the confined nature of nasal and ear passages and the risk of causing additional discomfort or injury to the sensitive tissue within these cavities, especially on young subjects.

Current devices often lack the finesse required to safely navigate and operate within the limited access of nasal and ear passages. Additionally, the variability in size, shape, and material of foreign objects (such as beads, small toy parts, or organic material) further complicates their removal. This can lead to multiple attempts at extraction, increasing the discomfort for the patient and the risk of mucosal damage.

Moreover, many existing tools do not provide a secure grip on smooth or irregularly shaped objects, leading to incomplete removal or the object slipping and potentially causing further complications. There is also a significant risk of the tools themselves causing trauma due to their rigid and sometimes sharp nature.

### SUMMARY

The present disclosure provides a device for foreign object extraction from a cavity of a subject. The device comprises (a) an insertion portion configured for being inserted into the cavity; (b) a reservoir configured to contain a fluidic activable adhesive substance, and for delivering the activable adhesive substance to a distal end of the insertion portion (i.e. so as to reach a front/facing surface of the foreign object when the distal end of the insertion portion is brought in contact of the foreign object); and (c) an adhesive substance actuator configured to controllably activate the adhesive substance so as to transition (cure) the adhesive substance from a non-adhesive state to an adhesive state (i.e. so as to generate bonding to the foreign object).

In addition to the above features, the device for foreign object extraction according to the present disclosure can optionally comprise one or more of features (i)-(xxi) below, in any technically possible combination or permutation:
(i) the insertion portion is configured to be inserted into an ear canal and/or nasal cavity.
(ii) the reservoir is arranged on the insertion portion.
(iii) the reservoir includes a spongious component arranged at a distal end of the insertion portion.
(iv) the spongious component is imbued with the activable substance.
(v) the activable adhesive substance is a photopolymer fluid configured to transition from the non-adhesive state to the adhesive state upon illumination in a predefined wavelength range;
(vi) the adhesive substance actuator includes a light source emitting in the predefined wavelength range configured to illuminate a distal end of the insertion portion.
(vii) the adhesive substance actuator is arranged on the insertion portion proximally to the reservoir.
(viii) the device is configured to be removably coupled to an endoscope so that the insertion portion protrudes longitudinally from a distal tip of the endoscope.
(ix) the insertion portion is configured so that a distal imaging capability of the endoscope is at least partially maintained through the insertion portion.
(x) the insertion portion comprises an opening or a transparent portion configured for at least partially maintaining the distal imaging capability of the endoscope.
(xi) the insertion portion further comprises:
   a. a proximal connecting section configured to removably couple to the distal tip of the endoscope; and
   b. a distal collar protruding distally from the proximal connecting section and configured to support the reservoir and the adhesive substance actuator;
wherein the adhesive substance actuator and the reservoir are coaxially arranged on the distal collar.
(xii) the proximal connecting section is configured as a sleeve to be coupled on the endoscope so as to partially surround (fit on) the distal tip of the endoscope.
(xiii) the proximal connecting section is configured as a fastening member configured to be fastened at the distal tip of the endoscope and includes a plurality of linking legs joining the proximal connecting section and the distal collar, wherein said linking legs extend from a peripheral portion of the collar to a peripheral portion of the proximal connecting section.
(xiv) the reservoir includes a tubular spongious component protruding axially from the distal collar and imbued with a photopolymer fluid configured to irreversibly transition from the non-adhesive state to the adhesive state upon illumination in a predefined wavelength range.
(xv) further comprising a protective sheath enveloping the spongious component.
(xvi) further comprising a retractile shroud configured for exposing a distal end of the tubular spongious component when a resisting axial force above a predefined threshold is applied to the shroud upon encountering a foreign object and for otherwise covering said tubular spongious component distal end.
(xvii) the adhesive substance actuator includes one or more light source emitting in said predefined wavelength range, said light source being carried on a ring sitting on the distal collar proximally to the reservoir.
(xviii) further comprising electric conductive pathways integrated within the sleeve configured for supplying power from connectors positioned on the sleeve to the one or more light source.
(xix) the connectors positioned on the sleeve are configured for detachable connection to an external power source.
(xx) wherein the external power source is operable by a trigger button, allowing for controlled activation of the one or more light source.
(xxi) manufactured from materials suitable for single-use operation and suitable to be discarded after one use.

The present disclosure also provides a method for extracting a foreign object from a cavity of a subject using a device, the method comprising: (a) inserting an insertion portion of the device into the cavity until contact with the foreign object, wherein the insertion portion is equipped with a reservoir containing an activable adhesive substance, while maintaining the activable adhesive substance in a non-adhesive state; (b) providing the activable adhesive substance at a distal end of the insertion portion and activating the activable adhesive substance by employing an adhesive substance actuator to adhere the activated adhesive substance to the foreign object, wherein activation transitions the adhesive substance from the non-adhesive state to an adhesive state; (c) retracting the insertion portion from the cavity, thereby extracting the adhered foreign object.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 is a block diagram illustrating generally a device for foreign object extraction from a cavity of a subject according to embodiments of the present disclosure;

FIG. 2A-2B illustrate an endoscope for imaging a subject's cavity according to embodiments of the present disclosure;

FIG. 3A-3F illustrate a device for foreign object extraction from a cavity of a subject and some of its components according to embodiments of the present disclosure;

FIG. 4A-4C illustrate a device for foreign object extraction from a cavity of a subject and some of its components according to embodiments of the present disclosure;

FIG. 5 illustrates a method for foreign object extraction from a cavity of a subject according to embodiments of the present disclosure.

### DETAILED DESCRIPTION

In the following detailed description, numerous specific details are set forth in order to provide a thorough understanding. However, it will be understood by those skilled in the art that the presently disclosed subject matter may be practiced without these specific details. In other instances, well-known methods and features have not been described in detail so as not to obscure the presently disclosed subject matter.

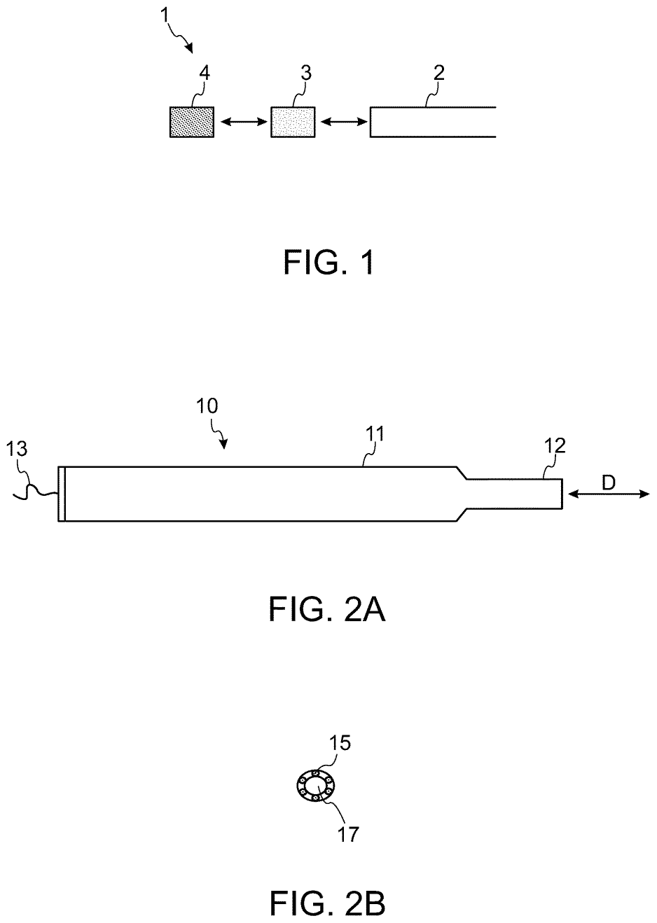

FIG. 1 illustrates generally a device 1 for foreign object extraction from a cavity (e.g. an aural cavity or a nasal cavity) of a subject. Mainly, the device 1 comprises an insertion portion 2, a reservoir 3 configured to contain an fluidic activable adhesive substance and an actuator 4 for controllably activate the activable adhesive substance. The device 1 may include a reservoir containing the activable adhesive substance or an “empty” reservoir adapted for being filled by said substance for example, prior to use. This may allow a caregiver to practice the extraction process without risks associated with accidentally touching the cavity walls with the activable adhesive substance. The device 1 may be manufactured from materials suitable for single-use operation and suitable to be discarded after one use. In some embodiments, only certain components of the device 1 such as the reservoir 3 may be disposable while the other components are reusable.

The insertion portion 2 may generally be cylindrical or slightly conical to match a shape of the ear canal. The insertion portion 2 may be configured so that it reaches no more than 15 mm inside the ear canal to prevent damage to deeper structures of the ear.

The insertion portion 2 may be configured for being inserted into the cavity. The insertion portion may have an elongated shape. The insertion portion 2 may form a distal part of the device 1. A distal end of the insertion portion 2 may be configured for preventing accidental scratching of the cavity. For example, the insertion portion may have a rounded profile to avoid corners and abrasive surfaces. The insertion portion 2 may be formed from biocompatible materials safe for contact with internal tissues such as medical-grade silicone or polymers. An external surface of the insertion portion 2 may be covered (or coated) to facilitate insertion and removal in the cavity.

The reservoir 3 may be configured to deliver the activable adhesive substance to a distal end of the insertion portion 2. The reservoir may be filled directly prior to use or during the manufacturing process (e.g. after assembly or before final packaging). For example, the activable adhesive substance may be a fluid and the reservoir 3 may be in fluid communication with the distal end of the insertion portion 2. In some embodiments, the device 1 may include a substance delivery conduit between the reservoir 3 and the distal end of the insertion portion 2. The substance delivery conduit may be controlled by a valve allowing precise amounts of the adhesive substance to be delivered to the distal end of the insertion portion 2. In some embodiments, the reservoir 3 may be arranged on the insertion portion 2, directly positioned at the distal end of the insertion portion 2. The insertion portion 2 may comprise a (central) distal support structure (e.g. a collar) configured for supporting the reservoir 3 and the adhesive substance actuator 4. The actuator 4 may be positioned proximal to the distal end of the insertion portion 2.

The activable adhesive substance may transition from a non-adhesive to an adhesive state when activated i.e. triggered by an external stimulus. Initially, in the non-adhesive state, the substance may be liquid or viscous and possess a slight tackiness (repositionable, pliable) which does not lead to strong or durable attachment to surfaces. This, allows for easy application and precise delivery. Activation (i.e. curing) of the adhesive may be achieved through light, temperature change, or chemical interaction for inducing a change in its structure, making it sticky. In the adhesive state, the substance maybe transformed into a hardened durable structure creating a strong and permanent bond with the surface it contacts (e.g. a front surface of the foreign object). The activable adhesive substance may typically be a photosensitive material (e.g. a photopolymer) sensitive to a specific light wavelength band e.g. ultraviolet light. The photopolymer may be formulated to undergo (rapid, e.g. from about 1 s to about 20 s, e.g. about 8 seconds) polymerization upon exposure to light in said light wavelength band. The light wavelength band may match an absorption spectrum of photo-initiators within the photopolymer to ensure efficient activation. The reservoir 3 may be a spongious component arranged at a distal end of the insertion portion 2. The reservoir 3 may be filled (imbued) with the activable adhesive substance. The filling may be performed directly prior to use (e.g. by injection into the spongious component) or during the manufacturing process (e.g. after assembly or before final packaging). In some embodiments, the spongious component is replaceable after use.

The transition from the non-adhesive state to the adhesive state may be controlled by the actuator 4. Upon transition, the substance forms a bond with the foreign object This bond is strong enough to support the extraction process but is also designed to minimize damage or irritation to any biological tissues involved. The adhesive substance may be such that the bond may sustain a pulling force from about 1 N to about 10 N, e.g. 4 N when glued to a clean glass surface. The adhesive substance may be biocompatible, posing no harmful effects when in contact with body tissues. The adhesive's formulation allows for controlled deployment and activation, meaning it does not prematurely bind to unwanted areas such as the device itself or the walls of the cavity. For applications where the adhesive should not remain permanently, the formulation might include properties that allow it to degrade under certain conditions—like another wavelength of light, exposure to a specific temperature, or a biological process. In other applications, the transition from the non-adhesive state to the adhesive state is non-reversible, at least in standard environmental conditions.

The adhesive substance actuator 4 may be configured to controllably activate the adhesive substance when delivered to the distal end of the insertion portion 2. Typically, in embodiments in which the activable adhesive substance is a photosensitive material such as a photopolymer, the actuator may include a UV light source such as one or more Light-Emitting Diodes (LEDs) emitting in the ultraviolet spectrum e.g. within the range of 200 nm to 400 nm. The UV light source may be configured to illuminate a distal end of the insertion portion. The UV light source may be configured to deliver an intensity of light suitable for activating the adhesive substance uniformly within a predefined time period and without overheating or damaging surrounding tissues. The light source may be configured so that a direction and spread of UV light is confined into a target area containing the distal end of the insertion portion where the adhesive substance is delivered by the reservoir. This may prevent unintended exposure of surrounding tissues.

In embodiments in which the reservoir 3 includes a spongious component imbued with the photopolymer arranged at a distal end of the insertion portion 2, the actuator 4 may include a UV light source arranged on the insertion portion 2 proximally to the reservoir 3 so as to illuminate the distal end of the spongious component intended to contact the foreign object.

The device 1 may be configured to be removably coupled to an endoscope (e.g. an otoscope, a rhinoscope, and/or a ENT endoscope) so that the insertion portion 2 (at least partially) protrudes longitudinally from a distal tip of the endoscope. The insertion portion 2 may in these embodiments include a proximal connecting section configured to removably couple to the distal tip of the endoscope and a central distal structure configured as a collar protruding distally from the proximal connecting section and configured to support the reservoir and the adhesive substance actuator. The adhesive substance actuator and the reservoir may be coaxially arranged on the distal collar. FIG. 2 illustrates a typical endoscope 10 for ear cavity navigation in some embodiments of the present disclosure. The endoscope 10 may resemble a pen in shape and dimensions for facilitating portable handling. The endoscope 10 may mainly be composed of a proximal cylindrical section 11 forming a handle of the endoscope 10 and a distal cylindrical section 12 forming a probe of the endoscope 10. A diameter of the handle 11 may be from about 5 mm to about 20 mm to ensure secure grip during examinations. A diameter of the probe 12 may be from about 2 mm to about 5 mm to ensure non-invasive access into the ear canal of a subject. The endoscope may include a camera module configured for capturing images of the ear canal. The camera module may include a lens assembly 17 configured for imaging a plane at a focus distance D from a distal end of the probe onto a sensor array. The focus distance may be from about 10 mm to about 20 mm. The device 1 may protrude from the endoscope 10 from a longitudinal dimension shorter than the focus distance (e.g. 15 mm). The device 1 may be configured to increase an external diameter of the endoscope by adding from about 0.5 mm to about 1.5 mm, e.g. about 1 mm. The camera module may further include a light source for providing illumination to enable visualization of the ear canal. The light source may include a plurality of LED 15 arranged at the distal end of the probe peripherally to the lens assembly 17. The endoscope may further include a light filter to ensure that the endoscope illumination does not activate the adhesive substance. The endoscope may further include an external connection 13 (e.g. a USB connection) configured to allow interfacing with a computing device (e.g. a smartphone or a laptop). This may enable real-time video streaming during foreign object extraction.

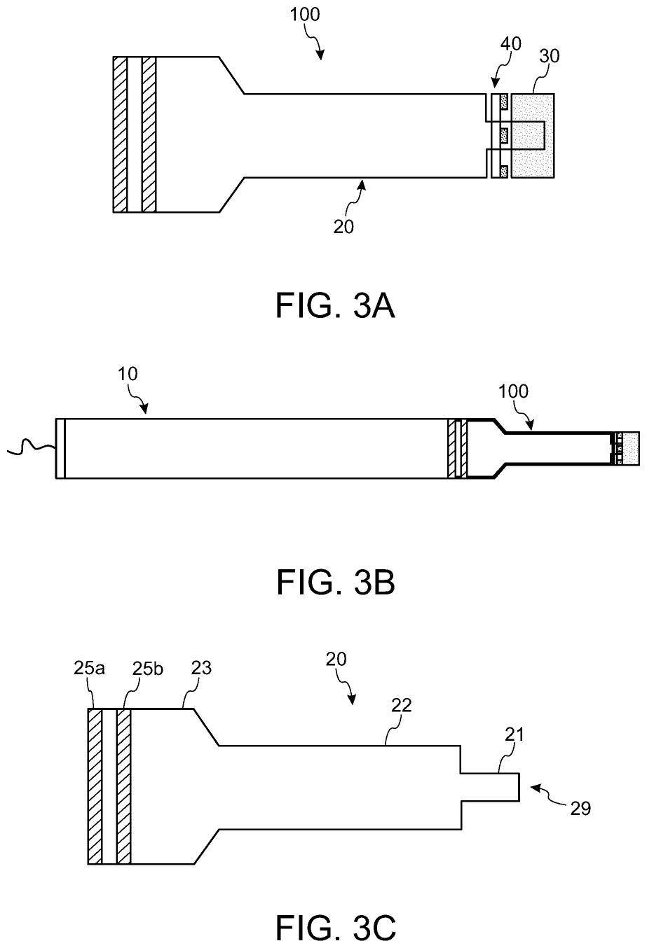

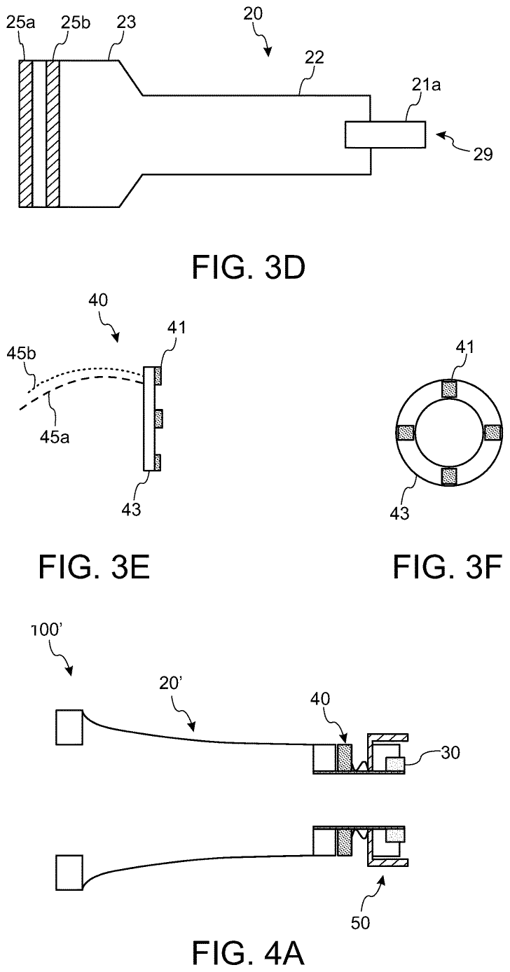

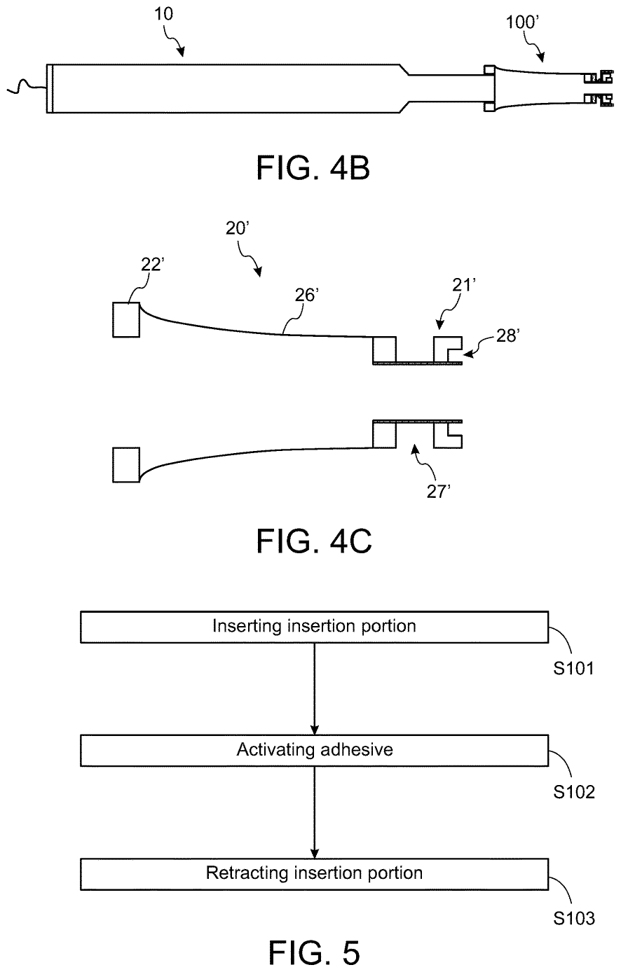

The device 1 may be coupled to the endoscope in various ways. In some embodiments described in more details hereinbelow with reference to FIGS. 3A-3F, the device may be configured as a form-fitting sleeve securely mounted on and enveloping the distal tip of the endoscope. In some embodiments, described in more details hereinbelow with reference to FIGS. 4A-4C, the device may be configured as a modular add-on configured to be fastened (e.g. clipped) at the distal tip of the endoscope and protrude therefrom.

Generally, the insertion portion 2 may be configured so that a distal imaging capability of the endoscope 10 is at least partially maintained through the insertion portion. For example, the insertion portion 2 may comprise an opening or a transparent portion (e.g. configured to face the lens assembly 17) for at least partially maintaining the distal imaging capability of the endoscope. For example, the central distal structure of the insertion portion 2 facing the lens assembly 17 may include a hollow tube or a transparent cylinder (which may form the distal collar) so that the endoscope can effectively image therethrough the body cavity. The transparent cylinder may have planar end surfaces. The transparent cylinder may be unitary with the rest of the insertion portion or be attached thereto. The transparent cylinder may form an interface with a different refraction index from the rest of the insertion portion. The transparent cylinder may protrude distally (so as to form a pin/collar) and/or proximally (so as to form a stopper for the endoscope lens assembly) from/into the distal tip of the insertion portion. For example, about 50% of the endoscope field of view may be maintained for enabling inspection. Furthermore, the actuator 4 may be triggered a manual control such as a foot pedal, a button or a switch. Therefore, as long as the trigger has not been pressed, the caregiver can pull the endoscope out without any gluing.

FIG. 5 generally describe a method of extracting a foreign object from a cavity of a subject using a device according to embodiments of the present disclosure. A first step S101 comprises inserting the insertion portion of the device into the subject's cavity. The reservoir may be preliminarily filled with the activable adhesive substance. The activable adhesive substance may remain in a non-adhesive state during this initial insertion in order to ensure that the adhesive does not inadvertently adhere to the cavity walls or other unintended surfaces. Upon successful positioning of the insertion portion adjacent to a front face of the foreign object, the method includes a further step S102. In this step, the activable adhesive substance present at the distal end of the insertion portion is activated by the adhesive substance actuator. The actuator triggers curing of the adhesive from a non-adhesive to an adhesive state, enabling it to firmly bond to the foreign object. Finally, step S103 comprises retracting the insertion portion from the cavity. During this retraction, the foreign object, now securely adhered to the end of the insertion portion via the activated adhesive, is effectively extracted from the cavity. The method ensures the foreign object is removed safely and efficiently, lowering any potential discomfort or harm to the subject.

Reference is now made to FIG. 3A-3E illustrating embodiments of the present disclosure. More particularly, FIG. 3A illustrates a lateral view of a device 100 configured as a sleeve configured to be securely mounted (e.g. by friction fitting) at a distal end of an endoscope and including an insertion portion 20, a reservoir 30 and an actuator 40; FIG. 3B illustrates a lateral view an endoscope 10 fit with the device 100 of FIG. 3A; FIG. 3C illustrates the insertion portion 20 of the device 100 shown on FIG. 3A; FIG. 3D illustrates a variant of the insertion portion 20 differing by a distal collar protruding proximally into the sleeve (Elements in FIG. 3D that are denoted with the same reference numbers as those in FIG. 3C represent similar components or features. For the sake of brevity, the description of similar components is not repeated below); FIGS. 3E-3F illustrate respectively a side view and a front view of the actuator 40 of the device 100 shown on FIG. 3A.

The insertion portion 20 may comprise a handle fitting section 23 (optional) configured to interface with the endoscope handle to ensure secure fitting. The insertion portion may further include a proximal connecting section 22 configured as a sleeve to interface with the endoscope probe (e.g. by at least partially surrounding it) and a distal collar 21 at the forefront of the device 100 configured to protrude longitudinally from the proximal connecting section 22. The distal collar 21, 21a may form a pin configured for distally securing the actuator 40 and the reservoir 30 while maintaining distal imaging capability of the endoscope imaging module through its distal tip 29. The distal collar 21, 21a may comprise a hollow tube or a solid cylinder. The hollow tube may include a distal cover surface or form an opening at its distal tip 29. The solid cylinder may be transparent and configured to allow imaging therethrough. The transparent cylinder may have planar end surfaces. The distal collar 21a may protrude proximally into the insertion portion (see FIG. 3D). A proximal end of the distal collar 21, 21a may form a stopper configured for abutting the endoscope. The distal collar may be unitary with the rest of the insertion portion or be attached thereto. The distal collar 21, 21a may form an interface with a different refraction index from the rest of the insertion portion. The reservoir 30 may have a tubular shape and may be configured so as to be securely coaxially arranged on the distal collar 21, 21a. The actuator 40 may have a ring shape and may be configured to be securely coaxially arranged on the distal collar 21, 21a proximally to the reservoir 30. The insertion portion 20, reservoir 30, and the actuator 40 may be securely attached one to the other (e.g. glued).

The reservoir 30 may comprise a spongious component protruding axially from the distal collar 21, 21a. The reservoir 30 may be imbued (i.e. impregnated) with a photopolymer fluid configured to irreversibly transition from the non-adhesive state to the adhesive state upon illumination in a predefined wavelength range (e.g. UV light). The reservoir 30 may further comprise a protective sheath encasing (wrapping) at least partially the spongious component. In embodiments in which the device is manufactured with a reservoir 30 filled with the photopolymer, the protective sheath may encase laterally and distally the spongious component. In other words, the protective sheath may form a seal around the spongious component. Prior to use, a distal end of the protective sheath may be removed (e.g. slit), exposing the tip of the spongious component. In embodiments in which the reservoir is filled directly before use, the protective sheath may encase only laterally the spongious component. Prior to use, the distal end of the spongious component may be provided (e.g. injected) with the photopolymer in the non-adhesive state. The protective sheath may act as a lateral barrier preventing the adhesive substance from inadvertently contacting the cavity walls during insertion or positioning of the device. When encasing completely the spongious component (i.e. laterally and distally), the protective sheath may also ensure the integrity of the activable adhesive substance contained within the spongious component, ensuring it remains effective until the moment of use.

The adhesive substance actuator 40 may be configured to control the activation of the adhesive substance. The actuator 40 may include one or more light source 41 (e.g. 3 or 4 UV LEDs) emitting in the predefined wavelength range capable of curing the photopolymer fluid contained in the spongious component. The one or more light source may be carried on a ring 43 sitting on the distal collar 21, 21a proximally to the reservoir 30. The light source may be positioned peripherally on a distal surface of the ring 43 so as to illuminate the distal end of the reservoir 30 (e.g. through the reservoir 30 itself). The actuator 40 may further include electric conductive pathways 45a, 45b (e.g. wires). The electric conductive pathways 45a, 45b may be integrated in the sleeve and configured for supplying power from connectors 25a, 25b positioned on the insertion portion 20 (e.g. on the handle fitting section 23) to the one or more light source 41. The connectors 25a, 25b may be configured for detachable connection to an external power source. The external power source may for example be operable by a trigger button or a foot pedal, allowing for controlled activation of the one or more light source.

Reference is now made to FIGS. 4A-4C illustrating embodiments of the present disclosure. Elements in FIGS. 4A-4C that are denoted with the same reference numbers as those in FIGS. 3A-3E represent similar components or features. For the sake of brevity, the description of similar components is not repeated below. More particularly, FIG. 4A illustrates a lateral cross section view of a device 100′ configured as an add-on device configured to be securely attached (e.g. by clipping, snapping, latching, threading and the like) at a distal end of an endoscope and including an insertion portion 20′, a reservoir 30 and an actuator 40; FIG. 4B illustrates a lateral view of an endoscope 10 attached with the device 100′ of FIG. 4A; FIG. 4C illustrates a lateral cross section view the insertion portion 20′ of the device 100′ shown on FIG. 4A.

The insertion portion 20′ may comprise a proximal connecting section 22′ configured as a fastening member to removably and securely attach to a tip of the endoscope probe (e.g. by clipping, snapping, latching, threading and the like). The a proximal connecting section 22′ may have a ring shape. The insertion portion 20′ may further comprise a distal collar 21′ at the forefront of the device 100′ configured to protrude longitudinally from the proximal connecting section 22′. The distal collar 21′ may have a tubular shape and form a proximal annular groove 27′ configured to receive the actuator 40 and a distal annular groove 28′ configured to receive the reservoir 30. The insertion portion 2 (and in particular the distal collar 21′) may include a central hollow for maintaining distal imaging capability of the endoscope imaging module. The reservoir 30 may have a tubular shape and may be configured so as to be securely coaxially arranged on the distal collar 21 in the distal annular groove 28′. The reservoir 30 may protrude distally out of the distal annular groove 28′. The actuator 40 may have a ring shape and may be configured to be securely coaxially arranged on the distal collar 21 in the proximal annular groove 27′. The insertion portion 20′ may further comprise a plurality of linking legs 26′ joining the proximal connecting section 22′ and the distal collar 21′. The linking legs 26′ may extend from a peripheral portion of the collar 21′ to a peripheral portion of the proximal connecting section 22′. The electric conductive pathways 45a, 45b of the actuator 40 may extend through the linking legs 26′. Connectors (not shown in this embodiment) may be positioned on the insertion portion 20 (e.g. on the proximal connecting section 22′) and the linking legs 26′ may be configured for supplying power from said connectors to the one or more light source 41.

Furthermore, the device may comprise a retractile shroud 50 configured for exposing a distal end of the reservoir 30 when a resisting axial force above a predefined threshold is applied to the retractile shroud 50 upon encountering a foreign object and for otherwise covering said tubular spongious component distal end. For example, the retractile shroud 50 may include a compression spring. In some examples, the spring of the retractile shroud may expose the distal end of the reservoir under a compression force from about 0.2 N to about 0.4 N, e.g. 0.3 N.

Those skilled in the art to which the present disclosure pertains, can appreciate that while the present disclosure has been described in terms of preferred examples, the concept upon which this disclosure is based may readily be utilized as a basis for the designing of other structures, systems and processes for carrying out the several purposes of the present disclosure.

Also, it is to be understood that the phraseology and terminology employed herein are for the purpose of description and should not be regarded as limiting. It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases. The term “each” may not be exclusively understood as referring to each and every, and when technically relevant may also refer to “at least some”.

All patents and patent applications mentioned in this specification are herein incorporated in their entirety by reference into the specification, to the same extent as if each individual patent or patent application was specifically and individually indicated to be incorporated herein by reference. In addition, citation or identification of any reference in this application shall not be construed as an admission that such reference is available as prior art to the present disclosure.

It is important, therefore, that the scope of the present disclosure is not construed as being limited by the illustrative examples set forth herein. Other variations are possible within the scope of the present disclosure as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1-22. (canceled)

23. A device for foreign object extraction from a cavity of a subject, comprising:
(a) an insertion portion configured for being inserted into the cavity;
(b) a reservoir configured to contain a fluidic activable adhesive substance, and for delivering the activable adhesive substance to a distal end of the insertion portion;
(c) an adhesive substance actuator configured to controllably activate the adhesive substance so as to transition the adhesive substance from a non-adhesive state to an adhesive state.

24. The device of claim 23, wherein the insertion portion is configured to be inserted into an ear canal and/or nasal cavity.

25. The device of claim 23, wherein the reservoir is arranged on the insertion portion.

26. The device of claim 23, wherein the reservoir includes a spongious component arranged at a distal end of the insertion portion.

27. The device of claim 26, wherein the spongious component is imbued with the activable substance.

28. The device of claim 23, wherein the activable adhesive substance is a photopolymer fluid configured to transition from the non-adhesive state to the adhesive state upon illumination in a predefined wavelength range and the adhesive substance actuator includes a light source emitting in the predefined wavelength range configured to illuminate a distal end of the insertion portion.

29. The device of claim 23, wherein the adhesive substance actuator is arranged on the insertion portion proximally to the reservoir.

30. The device of claim 23, wherein the device is configured to be removably coupled to an endoscope so that the insertion portion protrudes longitudinally from a distal tip of the endoscope.

31. The device of claim 30, wherein the insertion portion is configured so that a distal imaging capability of the endoscope is at least partially maintained through the insertion portion.

32. The device of claim 31, wherein the insertion portion comprises an opening or a transparent portion configured for at least partially maintaining the distal imaging capability of the endoscope.

33. The device of claim 30, wherein the insertion portion further comprises:
(a) a proximal connecting section configured to removably couple to the distal tip of the endoscope; and
(b) a distal collar protruding distally from the proximal connecting section and configured to support the reservoir and the adhesive substance actuator;
wherein the adhesive substance actuator and the reservoir are coaxially arranged on the distal collar.

34. The device of claim 33, wherein the proximal connecting section is configured as a sleeve to be coupled on the endoscope so as to partially surround the distal tip of the endoscope.

35. The device of claim 33, wherein the proximal connecting section is configured as a fastening member configured to be fastened at the distal tip of the endoscope and includes a plurality of linking legs joining the proximal connecting section and the distal collar, wherein said linking legs extend from a peripheral portion of the collar to a peripheral portion of the proximal connecting section.

36. The device of claim 33, wherein the reservoir includes a tubular spongious component protruding axially from the distal collar and imbued with a photopolymer fluid configured to irreversibly transition from the non-adhesive state to the adhesive state upon illumination in a predefined wavelength range.

37. The device of claim 36, further comprising a protective sheath enveloping the spongious component.

38. The device of claim 36, further comprising a retractile shroud configured for exposing a distal end of the tubular spongious component when a resisting axial force above a predefined threshold is applied to the shroud upon encountering a foreign object and for otherwise covering said tubular spongious component distal end.

39. The device of claim 33, wherein the adhesive substance actuator includes one or more light source emitting in said predefined wavelength range, said light source being carried on a ring sitting on the distal collar proximally to the reservoir.

40. The device of claim 39, further comprising electric conductive pathways integrated within the sleeve configured for supplying power from connectors positioned on the sleeve to the one or more light source.

41. The device of claim 40, wherein the connectors positioned on the sleeve are configured for detachable connection to an external power source.

42. The device of claim 41, wherein the external power source is operable by a trigger button, allowing for controlled activation of the one or more light source.
